(19) United States
(12) Patent Application Publication
(10) Pub. No.: US 2009/0277464 A1
(43) Pub. Date: Nov. 12, 2009

(54) Gravity Aided Restraint

(76) Inventor: Colleen K. CHAVEZ, Norwalk, CA (US)

Correspondence Address: GEOFFREY E. DOBBIN, PATENT ATTORNEY, 4278 SOUTH 6220 WEST, WEST VALLEY CITY, UT, 84128-6501, US

(21) Appl. No.: 12/118,662

(22) Filed: May 9, 2008

(51) Int. Cl. A61F 5/37

(52) U.S. Cl. 128/878

(57) ABSTRACT

The present invention is a gravity assisted passive restraint for use during surgery. The restraint comprises two hand-rings attached with a mostly inelastic connection band. The rings may take any shape, though round and “D” rings are preferred, and may feature finger grooves for added comfort and efficacy. The connection band may take any shape desired. Dimensions will be dependent upon desired location of the patient's limbs, either above or below the patient's diaphragm, and the size of the patient. In use, the patient's relaxed hands are positioned to hook into the hand rings while gravity pulls the hands away from each other, thus positioning the hands by using the tension generated in the band.

### FIELD OF THE INVENTION

The present invention relates to the field of surgical restraints and more particularly relates to a gravity aided restraint that is useful in positioning the hands and arms of a patient during medical and dental surgeries.

### BACKGROUND OF THE INVENTION

During surgery, a patient must be secured in order to minimize movement and keep limbs from getting in the way of the practitioner. As such, many restraints have been developed to secure a patient's arms during surgery. All of these restraints encompass the same two principals: immobilize the patient's limbs by either binding or encapsulating them. Binding restraints attach connected bands or cuffs about a patient's wrists. The bands/cuffs are connected by some form of cord, band, chain, or other structure that inhibits the independent movement of the patient's arms. Encapsulating restraints function by having a patient's arms slipped into a sleeve that is in a fixed position in relation to the operating table or other area. In either case, a patient cannot easily remove the restraints, or remove themselves from the restraints. This may be an acceptable situation when the patient is unconscious under the influence of general anesthetic, but could be problematic for those surgeries requiring only local anesthetic and the patient is conscious and aware of his surroundings.

What is needed then is a restraint that is passive, that is to say patient consented and initiated, and will retain the position of the arms in a relaxed position away from a surgical area. The restraint must also require minimal concentration and attention from the patient, yet still be effective. The present invention is a gravity assisted passive restraint that represents a departure from the prior art in that the restraint of the present invention allows for a patient to grip the restraint in a manner that utilizes gravity and the natural relaxed curvature of the patient's hands to secure the patient's limbs. In so doing, the restraint may be used in both local and general anesthesia surgeries and will not forcefully bind the patient.

### SUMMARY OF THE INVENTION

In view of the foregoing disadvantages inherent in the known types of surgical restraints, this invention provides a passive restraint that is assisted by gravity. As such, the present invention's general purpose is to provide a new and improved restraint that is easily used in both general and localized anesthesia while allowing the patient to not be physically bound.

To accomplish these objectives, the restraint comprises two hand rings and an inelastic connection between them. The patient merely holds the rings with palms facing inwards, towards the patient's chest. When relaxed, the natural curvature of the hand will hook into the rings and prevent the hands from slipping out of the rings, keeping the restraint and limbs in place. Various shapes and sizes may be utilized for different sized persons or for particular location of the limbs.

The more important features of the invention have thus been outlined in order that the more detailed description that follows may be better understood and in order that the present contribution to the art may better be appreciated. Additional features of the invention will be described hereinafter and will form the subject matter of the claims that follow.

Many objects of this invention will appear from the following description and appended claims, reference being made to the accompanying drawings forming a part of this specification wherein like reference characters designate corresponding parts in the several views.

Before explaining at least one embodiment of the invention in detail, it is to be understood that the invention is not limited in its application to the details of construction and the arrangements of the components set forth in the following description or illustrated in the drawings. The invention is capable of other embodiments and of being practiced and carried out in various ways. Also it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

### BRIEF DESCRIPTION OF THE DRAWINGS

FIG. 1 is a perspective view of one restraint according to the present invention.

FIG. 2 is a side plan view of the restraint of FIG. 1

FIG. 3 is a top plan view of the restraint of FIG. 1.

FIG. 4 is a side plan view of the restraint of FIG. 1 in use.

FIG. 5 is a top plan view of another restraint according to the present invention.

FIG. 6 is a top plan view of another restraint, for location of limbs in an upper body position, according to the present invention.

### DETAILED DESCRIPTION OF THE PREFERRED EMBODIMENTS

With reference now to the drawings, the preferred embodiment of the gravity aided restraint is herein described. It should be noted that the articles “a”, “an”, and “the”, as used in this specification, include plural referents unless the content clearly dictates otherwise.

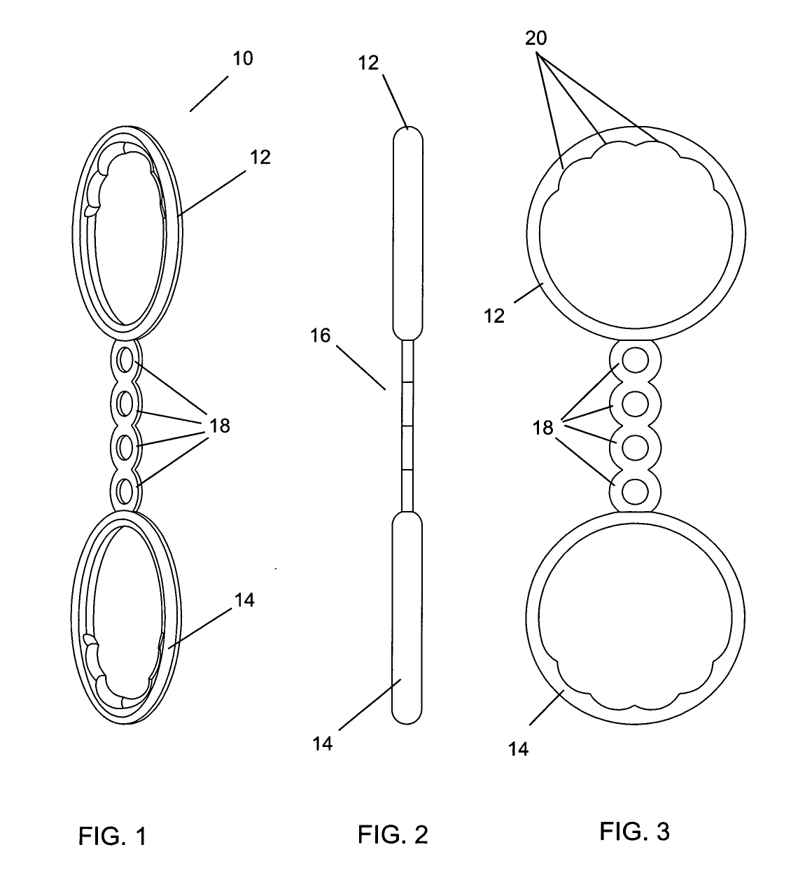

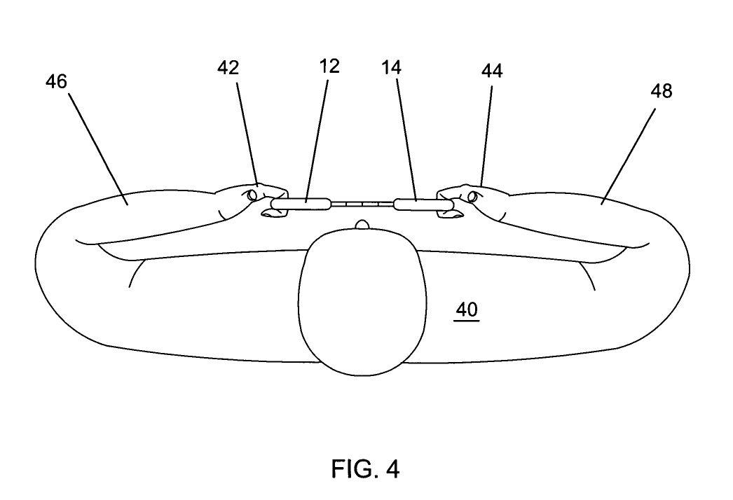

With reference to FIGS. 1-3, the restraint 10 comprises two solid hand rings 12, 14 and a connection band 16 between them. The band may take on any shape, such as the four rings 18 depicted in FIG. 2. Ideally, finger grooves 20 are provided to added comfort and friction, but these are not critical. Use is depicted in FIG. 4, where a patient's hands 42, 44 are partially inserted into the rings 12, 14. The hands are only inserted to the point where the fingers meet the palm and they are facing palm inwards. The band 16 is placed over the body 40. When relaxed, the hands naturally cup at that location, allowing relaxed hands 42, 44 to hook onto the rings 12, 14. The weight of the arms will then attempt to draw them apart an force the arms 46, 48 down to the patient's sides, however, the restraint 10 holds the arms and keeps them in position, out of the way of most surgeries. For those surgeries where this is not the case, another embodiment, shown in FIG. 6 is utilized. The alternate embodiment of FIG. 6 has a shorter band 66 and is positioned over the patient's chest. Due to the nature of the hooking action, a patient may or may not be conscious during the surgery. In either event, the restraint is easily held by a conscious patient with minimal effort and the position for most people is reasonably relaxed and comfortable. The restraint is easily held in place by a patient's natural reflexes is he is unconscious (under general anesthesia), and easily positioned and removed by surgical staff.

For an average to larger sized American, the hand-rings 12, 14 should have an inner height of about 5 inches (which may or may not be a true radius) and the restraint as a whole should measure almost 15 inches long. These measurements may of course be adjusted for different sized individuals (even larger patients and/or children). The materials from which the restraint should be made includes about any rubberized plastic or rubber that is suitable for a surgical environment, that is one that can be sterilized and is, for the most part, disposable. The choice of a rubberized plastic or rubber would then allow some give and flexibility in the hand rings for a more comfortable and secure hold. These materials are also relatively inexpensive, easy to manufacture and are recyclable. The rings are solid, incapable of opening, as that is not their function. If they were to be capable of opening, it is conceivable that a patient's hands may slip out of one or the other ring and ruin the purpose of the restraint. Solid rings are also easier to mold.

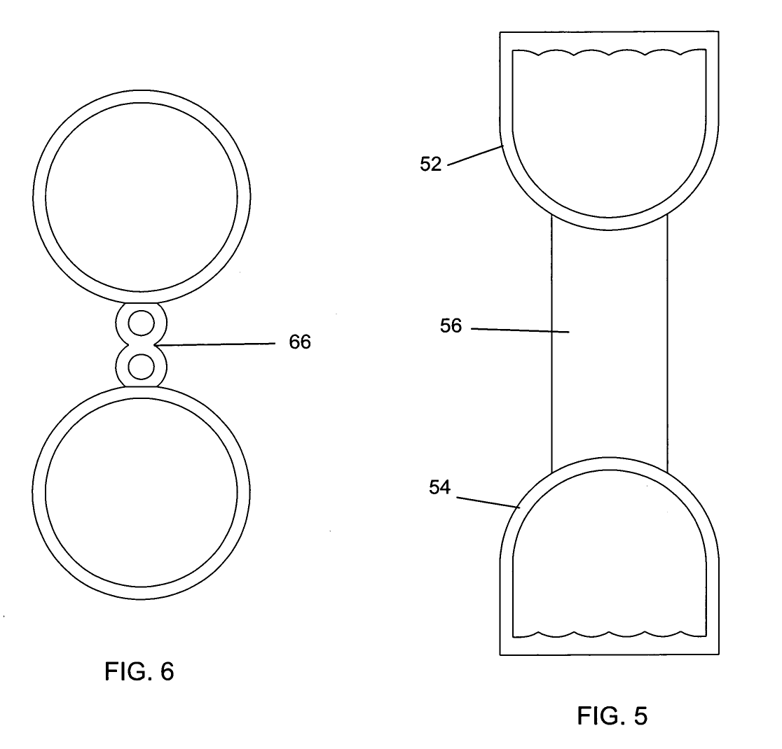

Other embodiments of the restraint include different shaped rings and bands, such as “D” rings 52, 54 and solid band 56 in FIG. 5. It should be noted that any shape and size of rings and band may be used so long as the sizing is sufficient to hold the arms against gravity. The band need only secure the rings so that the patient's arms are held with gravity. As such, the band need not be solid and may actually be a plurality of bands, rings or other shapes, or a lattice that would reduce material cost.

The restraint described above is, in particular, effective in those situations where the patient is conscious of the surgery (those requiring local anesthesia). This is particularly true in dentistry. The patient has their arms secured and can concentrate on holding the restraint, distracting from the surgery, or relax and forget about the restraint (and the surgery) as it will stay in position without effort by the patient. The materials from which the restraint is made also allow for comfortable and realistic gripping of the restraint (at the material will give somewhat) to relieve tension or aid in a procedure (like donating blood).

Although the present invention has been described with reference to preferred embodiments, numerous modifications and variations can be made and still the result will come within the scope of the invention. No limitation with respect to the specific embodiments disclosed herein is intended or should be inferred.

What is claimed is:

1. A gravity aided restraint comprising two solid hand rings and a connecting band therebetween, wherein the rings and connecting band are sized to secure a patient's arms against gravity.

2. The restraint to claim 1, further comprising finger grooves disposed on an interior side of each hand ring.

3. The restraint of claim 1 being made from a material selected from the group of materials consisting of: a rubberized plastic and rubber.

4. The restraint of claim 1, the hand rings being “D” shaped.

5. The restraint of claim 1, the hand rings being ellipses.

6. The restraint of claim 1 being sized to secure a patient's arms below the patient's diaphragm.

7. The restraint of claim 1 being sized to secure a patient's arms above the patient's diaphragm.
